(19) United States
(12) Patent Application Publication
(10) Pub. No.: US 2013/0299811 A1
(43) Pub. Date: Nov. 14, 2013

(54) DIBENZOTHIOPHENE DIOXIDE COMPOUND AND ORGANIC LIGHT-EMITTING DEVICE USING THE SAME

(71) Applicants: Masanori Seki, Yokohama-shi (JP); Kenichi Ikari, Abiko-shi (JP)

(72) Inventors: Masanori Seki, Yokohama-shi (JP); Kenichi Ikari, Abiko-shi (JP)

(73) Assignee: CANON KABUSHIKI KAISHA, Tokyo (JP)

(21) Appl. No.: 13/981,295

(22) PCT Filed: Dec. 9, 2011
(86) PCT No.: PCT/JP2011/079123

§ 371 Date: Jul. 23, 2013

(30) Foreign Application Priority Data
Jan. 25, 2011 (JP) 2011-012531

(51) Int. Cl. H01L 51/52

(52) U.S. Cl. CPC H01L 51/52
USPC 257/40; 549/46

(57) ABSTRACT

The present invention provides an organic light-emitting device showing characteristics of high efficiency and long operating life. The organic light-emitting device includes an anode, a cathode, and an organic compound layer disposed between the anode and the cathode. The organic compound layer includes at least a light-emitting layer that contains a dibenzothiophene dioxide compound shown in claim 1.

### TECHNICAL FIELD

The present invention relates to a dibenzothiophene dioxide compound and an organic light-emitting device using the same.

### BACKGROUND ART

An organic light-emitting device (organic electroluminescent device: organic EL device) is an electronic element including a pair of electrodes composed of an anode and a cathode and an organic compound layer disposed between these electrodes. Electrons and holes are injected from the pair of electrodes into the organic compound layer to generate excitons of the organic light-emitting compound in the organic compound layer, and the organic light-emitting device emits light when the excitons return to the ground state.

The organic light-emitting devices have remarkably progressed recently and are characterized by low driving voltages, various emission wavelengths, rapid response, and reduction in size and weight of light-emitting devices.

Compounds serving as constituent materials of the organic light-emitting devices have been actively being developed. For example, compounds having dibenzothiophene dioxides as the basic skeletons have been proposed as constituent materials of organic light-emitting devices. For example, Compound 1-A (see PTL 1) and Compound 1-B (see PTL 2) shown below have been proposed as fluorescent materials.

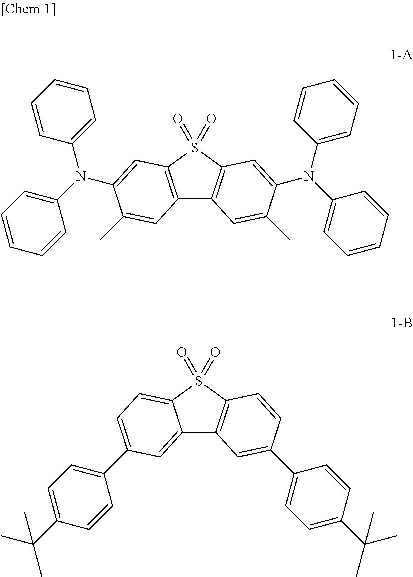 [SMILES: Cc1cc2c(cc1N(c1ccccc1)c1ccccc1)S(=O)(=O)c1cc(N(c3ccccc3)c3ccccc3)c(C)cc1-2 ; CC(C)(C)c1ccc(-c2ccc3c(c2)-c2cc(-c4ccc(C(C)(C)C)cc4)ccc2S3(=O)=O)cc1]

Compound 1-A disclosed in PTL 1 is a compound in which an amino group is introduced into a dibenzothiophene dioxide skeleton and is used as a constituent material of light-emitting layers or hole-transporting layers. Compound 1-B disclosed in PTL 2 is used as a constituent material of light-emitting layers, specifically, used as a fluorescent material.

### CITATION LIST

### Patent Literature

PTL 1 Japanese Patent No. 3114445
PTL 2 Japanese Patent Laid-Open No. 2001-313174

### SUMMARY OF INVENTION

However, Compounds 1-A and 1-B disclosed in PTLs 1 and 2 have not been proposed as constituent materials for phosphorescent light-emitting devices, for example, constituent materials of the light-emitting layers.

At the same time, organic compounds exhibiting hole-blocking and electron-injecting/transporting functions in organic light-emitting devices have been also actively being developed. Specifically, the compounds having the hole-blocking function and the electron-injecting/transporting functions that are used as constituent materials of a hole-blocking layer or an electron-transporting or injecting layer are required to have deep LUMO levels and be chemically stable. In particular, in organic light-emitting devices having light-emitting layers containing phosphorescent materials, high T₁ energies, as well as the above-mentioned LUMO levels and chemical stability, are necessary.

The present invention has been made solving the above-described problems and provides an organic light-emitting device showing characteristics of high efficiency and long operating life.

The dibenzothiophene dioxide compound according to the present invention is a compound represented by the following Formula [1] or [2].

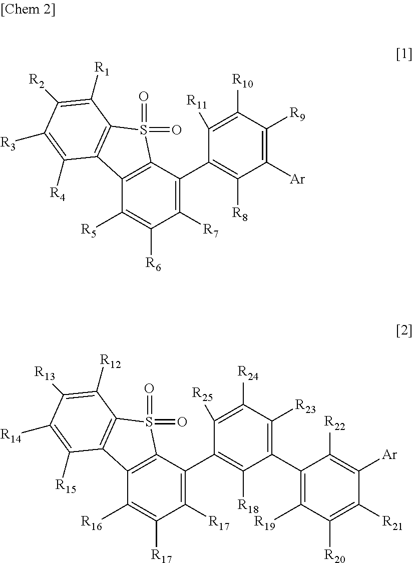

In Formulae [1] and [2], Ar represents a substituent selected from phenyl groups, terphenyl groups, phenanthryl groups, fluorenyl groups, and triphenylenyl groups and may optionally include an alkyl group having 1 to 4 carbon atoms as a substituent. In Formula [1], R₁ to R₁₁ each independently represent a hydrogen atom or an alkyl group having 1 to 4 carbon atoms. In Formula [2], R₁₂ to R₂₅ each independently represent a hydrogen atom or an alkyl group having 1 to 4 carbon atoms.

According to the present invention, an organic light-emitting device having characteristics of high efficiency and long operating life can be provided.

### BRIEF DESCRIPTION OF DRAWING

FIG. 1 is a schematic cross-sectional view illustrating an example of a display apparatus having organic light-emitting devices according to an embodiment of the present invention and TFT devices, as an example of switching devices, electrically connected to the organic light-emitting devices.

### DESCRIPTION OF EMBODIMENT

The dibenzothiophene dioxide compound according to the present invention is represented by the following Formula [1] or [2].

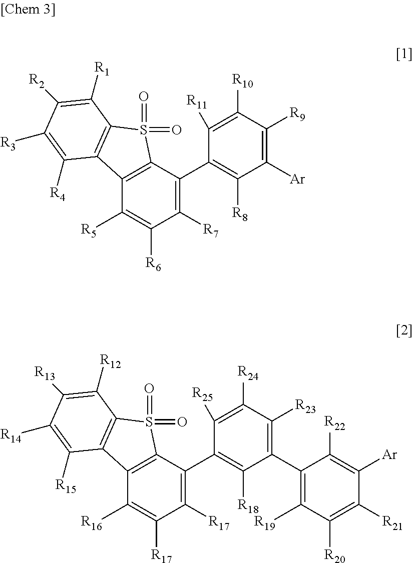

In Formulae [1] and [2], Ar represents a substituent selected from phenyl groups, terphenyl groups, phenanthryl groups, fluorenyl groups, and triphenylenyl groups.

The Ar may optionally include an alkyl group having 1 to 4 carbon atoms as a substituent, specifically, a substituent selected from methyl groups, ethyl groups, n-propyl groups, iso-propyl groups, n-butyl groups, iso-butyl groups, sec-butyl groups, and tert-butyl groups.

In Formula [1], R₁ to R₁₁ each independently represent a hydrogen atom or an alkyl group having 1 to 4 carbon atoms.

Examples of the alkyl groups represented by R₁ to R₁₁ include substituents selected from methyl groups, ethyl groups, n-propyl groups, iso-propyl groups, n-butyl groups, iso-butyl groups, sec-butyl groups, and tert-butyl groups.

In Formula [2], R₁₂ to R₂₅ each independently represent a hydrogen atom or an alkyl group having 1 to 4 carbon atoms.

Examples of the alkyl groups represented by R₁₂ to R₂₅ include substituents selected from methyl groups, ethyl groups, n-propyl groups, iso-propyl groups, n-butyl groups, iso-butyl groups, sec-butyl groups, and tert-butyl groups.

A method of synthesizing the organic compound (dibenzothiophene dioxide compound) according to the present invention will now be described below. The organic compound according to the present invention can be synthesized in accordance with, for example, the following synthetic scheme.

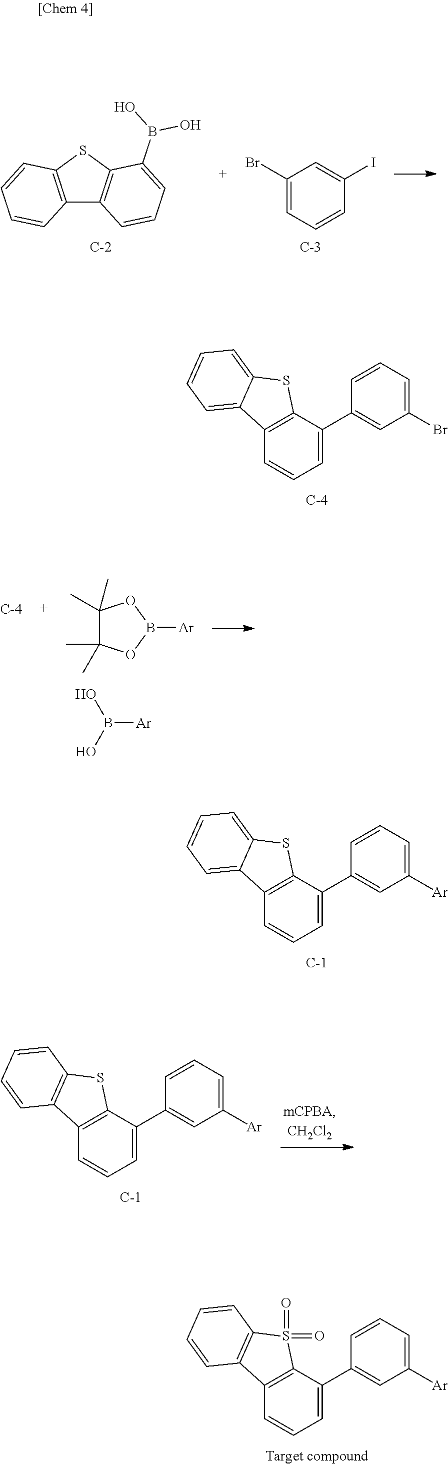

The organic compound according to the present invention, that is, the target compound shown in the synthetic scheme is synthesized through the following processes (a) to (c):

(a) a reaction between Compound C-2 (boronic acid) and Compound C-3 (synthesis of Compound C-4),
(b) a reaction between Compound C-4 and Compound C-5 (boronic acid) (synthesis of Compound C-1), and
(c) an oxidation reaction of Compound C-1 (synthesis of target compound).

The processes (a) and (b) are performed, for example, in a solvent mixture of toluene, ethanol, and distilled water in the presence of sodium carbonate and a catalyst (e.g., Pd(PPh₃)₄). The process (c) is a reaction oxidizing a sulfur atom (—S—), for example, with m-chloroperbenzoic acid in dichloromethane.

In the case of synthesizing the compound according to the present invention by the above-described synthetic scheme, various organic compounds can be synthesized by appropriately changing Compound C-5. Similarly, Example Compounds shown in Group B, which will be described below, can be synthesized by using 3-bromo-3′-chloro-1,1′-biphenyl instead of Compound C-3.

Next, characteristics of the organic compound (dibenzothiophene dioxide compound) according to the present invention will be described.

In the dibenzothiophene dioxide skeleton serving as the main skeleton of the organic compound according to the present invention, dibenzothiophene is oxidized at the 5-position (sulfur atom) as shown below.

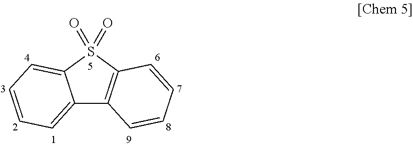 [SMILES: O=S1(=O)c2ccccc2-c2ccccc21]

The oxidation of the sulfur atom deepens the HOMO level and the LUMO level. Consequently, the hole-blocking properties and the electron-injecting and transporting properties of the organic compound according to the present invention are increased.

The HOMO level and the LUMO level were actually determined by molecular orbital calculation at the B3LYP/6-31G* level using density functional theory. The results were that the LUMO level of dibenzothiophene dioxide was deep such as −1.81 eV, whereas that of dibenzothiophene was −0.95 eV. This deep LUMO level is due to a sulfone group, and the dibenzothiophene dioxide skeleton having the sulfone group is a suitable skeleton as an electron-injecting/transporting material. Similarly, dibenzothiophene dioxide had a deep HOMO level of −6.67 eV whereas the HOMO level of dibenzothiophene was −5.82 eV. Consequently, the organic compound having a dibenzothiophene dioxide skeleton according to the present invention is suitable as a hole-blocking material.

In the case of using a phosphorescent material as the light-emitting material contained in a light-emitting layer and using the organic compound according to the present invention as the constituent material of a light-emitting layer or a layer (hole-blocking layer, electron-transporting layer) adjacent to a light-emitting layer, the T₁ energy of the organic compound according to the present invention is important.

Table 1 shows calculated values (B3LYP/6-31G* levels) of T₁ energies of dibenzothiophene dioxide and main fused rings.

TABLE 1

| | | T₁ energy |
|---|---|---|
| | Structural | (wavelength) |
| Name | formula | equivalent) |
| Dibenzothiphene dioxide | 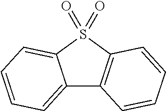 [SMILES: O=S1(=O)c2ccccc2-c2ccccc21] | 420 nm |
| Benzene | 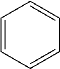 | 327 nm |
| Naphthalene | 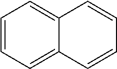 | 454 nm |
| Fluorene | 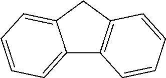 [SMILES: c1ccc2c(c1)Cc1ccccc1-2] | 401 nm |
| Phenanthrene | 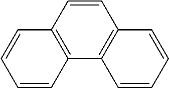 [SMILES: c1ccc2c(c1)ccc1ccccc12] | 453 nm |
| Triphenylene | 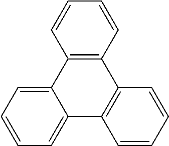 [SMILES: c1ccc2c(c1)c1ccccc1c1ccccc21] | 434 nm |

As shown in Table 1, dibenzothiophene dioxide, which is a compound serving as the basic skeleton of the organic compound according to the present invention, has a high T₁ energy. Therefore, the organic compound according to the present invention having a specific substituent introduced in the dibenzothiophene dioxide also has a high T₁ energy.

When the color of light emitted by a phosphorescent material is in a range of blue to green, that is, the maximum peak of an emission wavelength spectrum is in a range of 440 to 520 nm, the aryl group that is introduced into the organic compound according to the present invention, that is, the Ar shown in Formula [1] and [2] is restricted. Specifically, the Ar shown in Formula [1] and [2] is, for example, a phenyl group, a terphenyl group, a fluorenyl group, a phenanthryl group, or a triphenylenyl group. These aryl groups are substituents having high T₁ energies.

In the organic compound according to the present invention, the dibenzothiophene dioxide skeleton having a high T₁ energy and the aryl group having a high T₁ energy are linked to each other with a linker having a m-phenylene group or a m-biphenylene group. By using the m-phenylene group or the m-biphenylene group as a linker, extension of conjugation is prevented to give a compound having a high T₁ energy.

Introduction of a diarylamine skeleton into the 3-position or 7-position of the dibenzothiophene dioxide skeleton reduces the T₁ energy, and such a compound is not suitable as a constituent material of the phosphorescent light-emitting device that emits blue to green light. For example, the calculated T₁ energy of Compound I-A described in PTL 1 is low such as 523 nm. The arylamine skeleton has a function of reducing the HOMO level of a compound. Accordingly, introduction of an arylamine skeleton is suitable for a compound that is used as the constituent material of a hole-transporting layer, but is not suitable for a compound that is used as the constituent material of a hole-blocking layer or an electron-transporting layer that is in contact with a light-emitting layer.

Compound 1-B described in PTL 2 has substituents at the 2-position and the 8-position of the dibenzothiophene dioxide skeleton. Compound 1-B and Compound a (Example Compound B8, which belongs to the compound according to the present invention) were compared for their LUMO levels and T₁ energies. The LUMO levels were determined by measuring the band gap from the absorption edge of UV spectrum and adding the band gap to the HOMO level. The T₁ energy was determined from the result of the measurement of phosphorescent emission spectrum in a thin film form. The results are shown in Table 2.

TABLE 2

| Compound | Structure | LUMO level | T₁ energy (thin film) |
|---|---|---|---|
| 1-B | 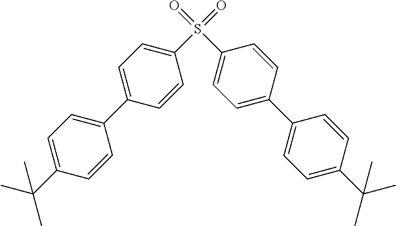 [SMILES: CC(C)(C)c1ccc(-c2ccc(S(=O)(=O)c3ccc(-c4ccc(C(C)(C)C)cc4)cc3)cc2)cc1] | −2.69 eV | 525 nm |
| a | 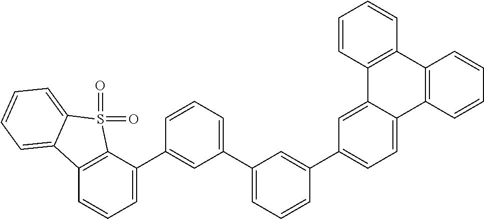 [SMILES: O=S1(=O)c2ccccc2-c2cccc(-c3cccc(-c4cccc(-c5ccc6c7ccccc7c7ccccc7c6c5)c4)c3)c21] | −3.03 eV | 500 nm |

Table 2 shows that Compound 1-B has a LUMO level shallower than that of Compound a and is not suitable as a constituent material for the hole-blocking layer or the electron-transporting layer that is in contact with a light-emitting layer. In addition, Table 2 shows that the T₁ energy of Compound 1-B is lower than that of Compound a, which belongs to the organic compound according to the present invention. This means that the T₁ energy of a compound having substituents introduced at the 2-position and 8-position of the dibenzothiophene dioxide skeleton is lower than that of a compound having a substituent at the 4-position (or 6-position) of the dibenzothiophene dioxide skeleton. Furthermore, Compound 1-B is not suitable as a constituent material for phosphorescent light-emitting devices that emit blue or green light, because of its T₁ energy level.

Accordingly, from the viewpoints of the LUMO level, maintenance of the T₁ energy, and stability of the compound, the substituent is introduced to the 4-position (or 6-position) of the dibenzothiophene dioxide skeleton.

Thus, an organic light-emitting device exhibiting high efficiency can be obtained by using the organic compound according to the present invention as a constituent material of a phosphorescent light-emitting device that emits blue or green light, in particular, by using the organic compound as a hole-blocking material, an electron-transporting material (electron-injecting material), or a host material.

Specific examples of the organic compound (dibenzothiophene dioxide compound) according to the present invention are shown below, but the present invention is not limited thereto.

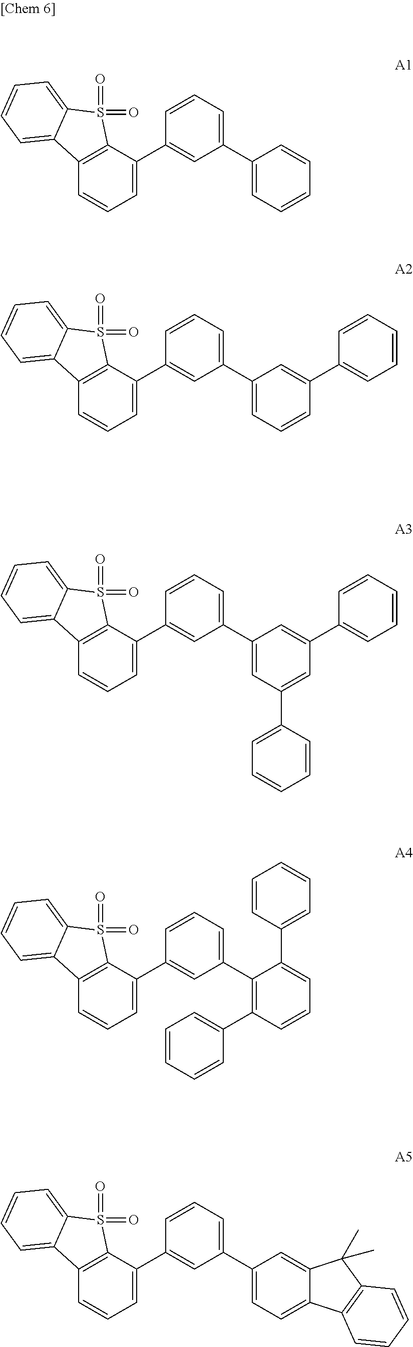 [SMILES: O=S1(=O)c2ccccc2-c2cccc(-c3cccc(-c4ccccc4)c3)c21 ; O=S1(=O)c2ccccc2-c2cccc(-c3cccc(-c4cccc(-c5ccccc5)c4)c3)c21 ; O=S1(=O)c2ccccc2-c2cccc(-c3cccc(-c4cc(-c5ccccc5)cc(-c5ccccc5)c4)c3)c21 ; O=S1(=O)c2ccccc2-c2cccc(-c3cccc(-c4c(-c5ccccc5)cccc4-c4ccccc4)c3)c21 ; CC1(C)c2ccccc2-c2ccc(-c3cccc(-c4cccc5c4S(=O)(=O)c4ccccc4-5)c3)cc21]

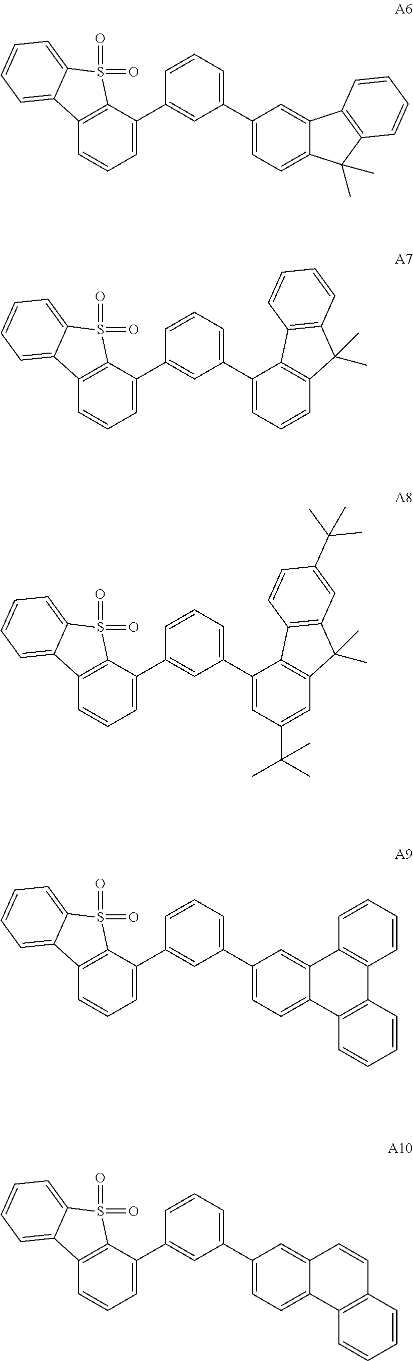 [SMILES: CC1(C)c2ccccc2-c2cc(-c3cccc(-c4cccc5c4S(=O)(=O)c4ccccc4-5)c3)ccc21 ; CC1(C)c2ccccc2-c2c(-c3cccc(-c4cccc5c4S(=O)(=O)c4ccccc4-5)c3)cccc21 ; CC(C)(C)c1ccc2c(c1)C(C)(C)c1cc(C(C)(C)C)cc(-c3cccc(-c4cccc5c4S(=O)(=O)c4ccccc4-5)c3)c1-2 ; O=S1(=O)c2ccccc2-c2cccc(-c3cccc(-c4ccc5c6ccccc6c6ccccc6c5c4)c3)c21 ; O=S1(=O)c2ccccc2-c2cccc(-c3cccc(-c4ccc5c(ccc6ccccc65)c4)c3)c21]

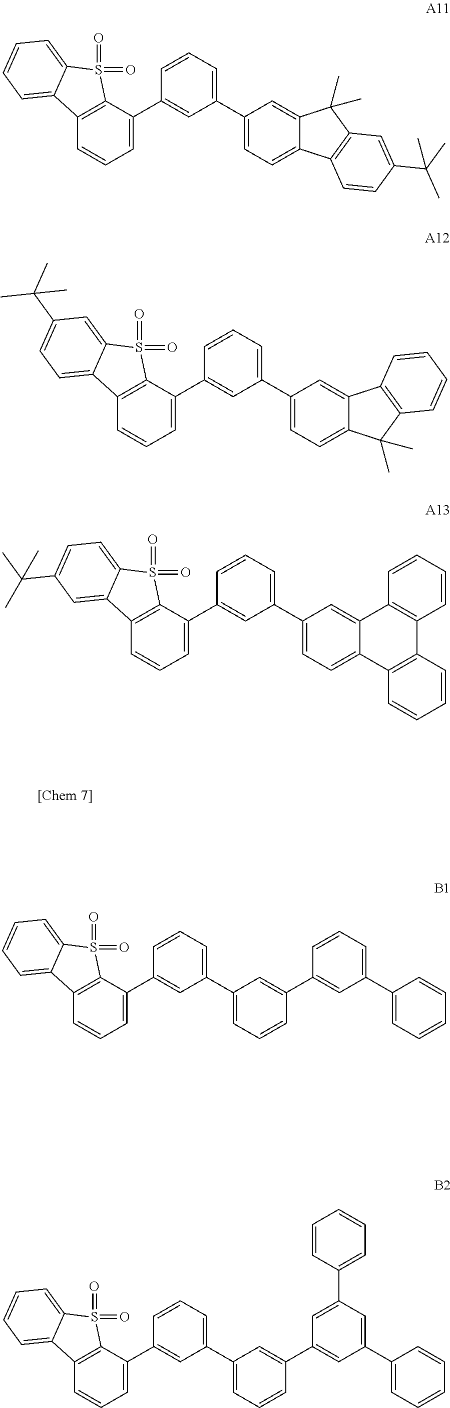

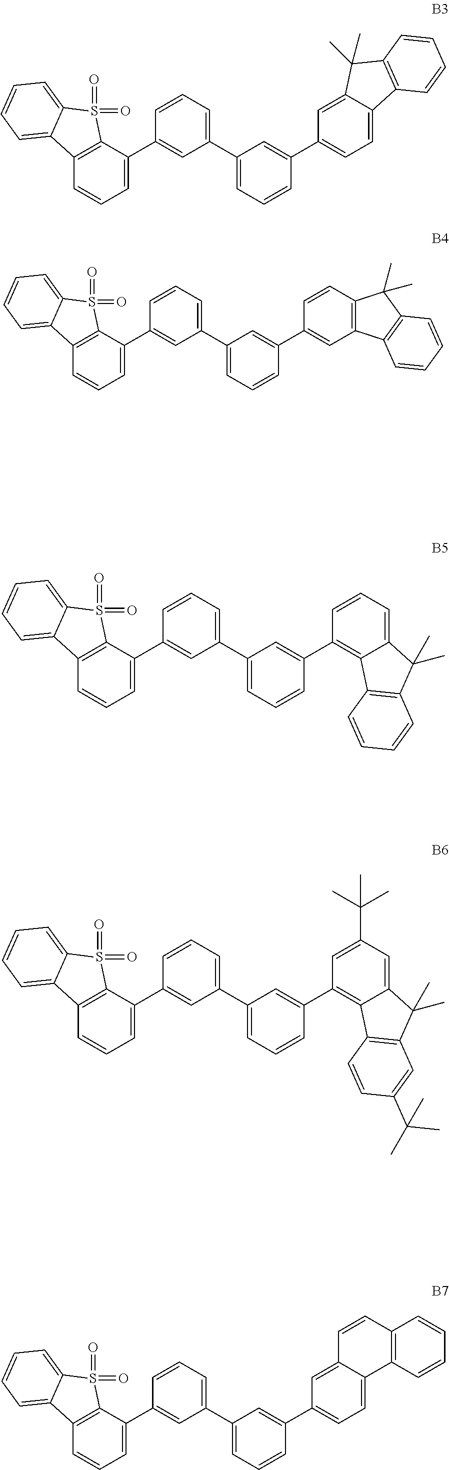 [SMILES: CC1(C)c2ccccc2-c2ccc(-c3cccc(-c4cccc(-c5cccc6c5S(=O)(=O)c5ccccc5-6)c4)c3)cc21 ; CC1(C)c2ccccc2-c2cc(-c3cccc(-c4cccc(-c5cccc6c5S(=O)(=O)c5ccccc5-6)c4)c3)ccc21 ; CC1(C)c2ccccc2-c2c(-c3cccc(-c4cccc(-c5cccc6c5S(=O)(=O)c5ccccc5-6)c4)c3)cccc21 ; CC(C)(C)c1ccc2c(c1)C(C)(C)c1cc(C(C)(C)C)cc(-c3cccc(-c4cccc(-c5cccc6c5S(=O)(=O)c5ccccc5-6)c4)c3)c1-2 ; O=S1(=O)c2ccccc2-c2cccc(-c3cccc(-c4cccc(-c5ccc6c(ccc7ccccc76)c5)c4)c3)c21]

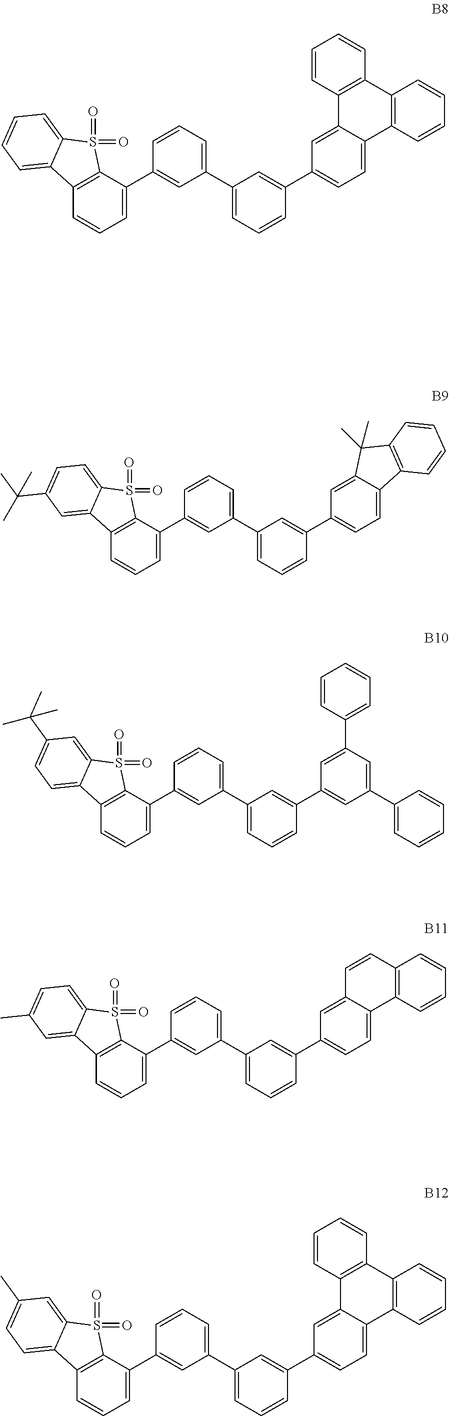 [SMILES: O=S1(=O)c2ccccc2-c2cccc(-c3cccc(-c4cccc(-c5ccc6c7ccccc7c7ccccc7c6c5)c4)c3)c21 ; CC(C)(C)c1ccc2c(c1)-c1cccc(-c3cccc(-c4cccc(-c5ccc6c(c5)C(C)(C)c5ccccc5-6)c4)c3)c1S2(=O)=O ; CC(C)(C)c1ccc2c(c1)S(=O)(=O)c1c(-c3cccc(-c4cccc(-c5cc(-c6ccccc6)cc(-c6ccccc6)c5)c4)c3)cccc1-2 ; Cc1ccc2c(c1)-c1cccc(-c3cccc(-c4cccc(-c5ccc6c(ccc7ccccc76)c5)c4)c3)c1S2(=O)=O ; Cc1ccc2c(c1)S(=O)(=O)c1c(-c3cccc(-c4cccc(-c5ccc6c7ccccc7c7ccccc7c6c5)c4)c3)cccc1-2]

Among the example compounds, the compounds belonging to Group A correspond to specific examples of the organic compound represented by Formula [1]. In the organic compounds represented by Formula [1], the dibenzothiophene dioxide skeleton and the Ar are linked to each other with a m-phenylene group. This m-phenylene group functions so as to disconnect the conjugation between the dibenzothiophene dioxide skeleton and the aryl moiety represented by Ar.

The compounds belonging to Group B correspond to specific examples of the organic compound represented by Formula [2]. In the organic compounds represented by Formula [2], the dibenzothiophene dioxide skeleton and the Ar are linked to each other with a m-biphenylene group. This m-biphenylene group functions so as to disconnect the conjugation between the dibenzothiophene dioxide skeleton and the aryl moiety represented by Ar.

Accordingly, the wavelength of T₁ energy of the organic compound according to the present invention is shorter than 490 nm.

The aryl group represented by Ar in Formulae [1] and [2] are selected from substituents having T₁ energies in the wavelength range of shorter than 490 nm, which is a characteristic of the organic compound according to the present invention. Specifically, the aryl group is selected from phenyl groups, terphenyl groups, phenanthryl groups, fluorenyl groups, and triphenyl groups. These aryl groups may have another substituent as long as the requirement that the wavelength of the T₁ energy is shorter than 490 nm is satisfied. Specifically, the aryl group may be further substituted with any of the above-mentioned alkyl groups having 1 to 4 carbon atoms.

The organic compound according to the present invention can be used as a constituent material of an organic light-emitting device, specifically, as a constituent material of a hole-blocking layer and also may be used as a constituent material of, for example, a light-emitting layer or an electron-injecting layer (electron-transporting layer). The organic compound according to the present invention may be used not only in a phosphorescent light-emitting device that emits green light but also in a phosphorescent light-emitting device that emits blue light. Even in the case of using the organic compound of the present invention in the phosphorescent light-emitting device that emits blue light, the compound can be used as the constituent material of a hole-blocking layer, a light-emitting layer, or an electron-injecting layer (electron-transporting layer).

In the case of using the organic compound according to the present invention as the constituent material of a hole-blocking layer or an electron-injecting layer (electron-transporting layer), the organic compound of the present invention can be used in any organic light-emitting device regardless of the color of the emitted light, in phosphorescent light-emitting devices and fluorescent light-emitting devices. For example, the organic compound of the present invention can be used as the constituent material of a blue light-emitting device, a bluish-green light-emitting device, a light-blue light-emitting device, a green light-emitting device, a yellow light-emitting device, an orange light-emitting device, a red light-emitting device, and a white light-emitting device.

An organic light-emitting device according to this embodiment will be described below.

The organic light-emitting device according to this embodiment includes a pair of electrodes composed of an anode and a cathode and an organic compound layer disposed between the anode and the cathode. In the organic light-emitting device according to the embodiment, the organic compound layer contains the organic compound according to the present invention. Furthermore, in the organic light-emitting device according to the embodiment, the organic compound layer is a monolayer or a laminate of a plurality of layers having at least a light-emitting layer.

Examples of the structure of the organic light-emitting device according to the embodiment include the following (i) to (v):

(i) (substrate/)anode/light-emitting layer/cathode,
(ii) (substrate/)anode/hole-transporting layer/electron-transporting layer/cathode,
(iii)(substrate/)anode/hole-transporting layer/light-emitting layer/electron-transporting layer/cathode,
(iv) (substrate/)anode/hole-injecting layer/hole-transporting layer/light-emitting layer/electron-transporting layer/cathode, and
(v)(substrate/)anode/hole-transporting layer/light-emitting layer/hole-blocking layer/electron-transporting layer/cathode.
Note that in the structure (ii), the hole-transporting layer, the electron-transporting layer, or the interface between the hole-transporting layer and the electron-transporting layer also has a function as a light-emitting layer.

The above-mentioned five specific structures are only basic device configurations, and the organic light-emitting device according to the embodiment is not limited these configurations.

When the organic light-emitting device according to the embodiment has a hole-blocking layer as shown in the above-mentioned structure (v), the organic compound according to the present invention can be used as the constituent material of the hole-blocking layer. This is because the organic compound according to the present invention has a high T₁ energy and can inhibit leakage of excitons generated in the light-emitting layer. This is particularly effective in a phosphorescent light-emitting device that emits green light, but the structure can be also applied to organic light-emitting devices that emit light of other colors.

The dibenzothiophene dioxide, which is the basic skeleton of the organic compound according to the present invention, is electron-withdrawing and therefore has characteristics of a deep LUMO level and high electron-transporting ability. Accordingly, the organic compound according to the present invention may be used as the constituent material of an electron-injecting layer or an electron-transporting layer. In the case of using the organic compound according to the present invention as the constituent material of an electron-injecting layer or an electron-transporting layer, the layer containing the organic compound according to the present invention may be further doped with an alkali metal such as lithium or cesium, an alkaline earth metal such as calcium, or a salt thereof.

The present inventors have performed various investigations and, as a result, have found that an organic light-emitting device can have a high efficiency by using the organic compound according to the present invention as a constituent material of a hole-blocking layer or an electron-injecting layer (electron-transporting layer). This is because the organic compound according to the present invention has a high T₁ energy to show a high property for transporting electrons and a high property for injecting electrons to a light-emitting layer. Consequently, holes and electrons can be efficiently recombined in the light-emitting layer.

In the organic light-emitting device according to the embodiment, the organic compound according to the present invention may be used as a host or guest material of a light-emitting layer. The guest material is a component that is contained in a light-emitting layer, defines the substantial color of light emitted by the organic light-emitting device, and emits light by itself. The host material is a component contained in a light-emitting layer in a composition ratio larger than that of the guest material. The composition ratio of the guest material in a light-emitting layer is low whereas the composition ratio of the host material in the light-emitting layer is high. The term “composition ratio” refers to a value calculated by using the total amount of components constituting a light-emitting layer as the denominator and shown by wt %.

In the organic light-emitting device according to the embodiment, the organic compound according to the present invention is particularly useful as the host material for a phosphorescent light-emitting layer. Specifically, in the case of combination with a guest material (phosphorescent light-emitting material) that emits light having a luminescence peak in a range of 440 to 660 nm in a blue to red range, the loss of triplet energy is small to increase the efficiency of the light-emitting device.

In the case of using the organic compound according to the present invention as a guest material of a light-emitting layer, the amount of the guest material relative to the amount of the host material can be 0.1 wt % or more and 30 wt % or less, such as 0.5 wt % or more and 10 wt % or less, based on the total amount of the materials contained in the light-emitting layer.

The organic light-emitting device according to the embodiment can optionally contain, for example, a known low-molecular or high-molecular hole-injecting material, hole-transporting material, host material, guest material, electron-injecting material, or electron-transporting material, together with the organic compound according to the present invention.

Examples of these compounds will be shown below.

As the hole-injecting material or the hole-transporting material, a material having high hole mobility can be used. Examples of the low- or high-molecular material having hole-injecting or transporting ability include, but not limited to, triarylamine derivatives, phenylenediamine derivatives, stilbene derivatives, phthalocyanine derivatives, porphyrin derivatives, poly(vinylcarbazole), poly(thiophene), and other electrically conductive polymers.

Examples of the host material contained in a light-emitting layer include, but not limited to, triarylamine derivatives, phenylene derivatives, condensed ring aromatic compounds (e.g., naphthalene derivatives, phenanthrene derivatives, fluorene derivatives, and chrysene derivatives), organic metal complexes (e.g., organic aluminum complexes such as tris(8-quinolinolate)aluminum, organic beryllium complexes, organic iridium complexes, and organic platinum complexes), and polymer derivatives such as poly(phenylenevinylene) derivatives, poly(fluorene) derivatives, poly(phenylene) derivatives, poly(thienylenevinylene) derivatives, and poly(acetylene) derivatives.

Examples of the guest material contained in a light-emitting layer include, but not limited to, phosphorescent Ir complexes shown below and platinum complexes.

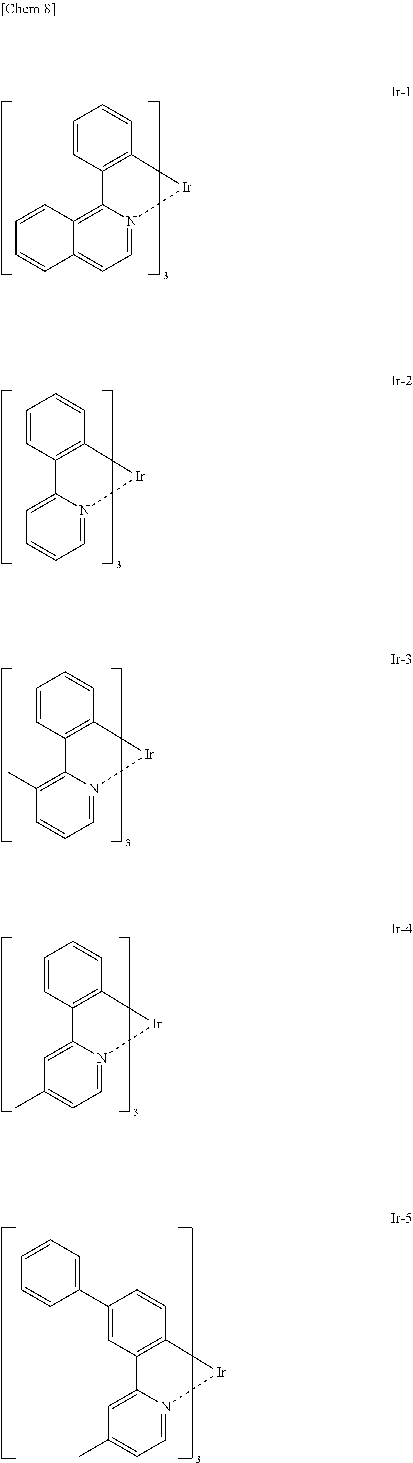

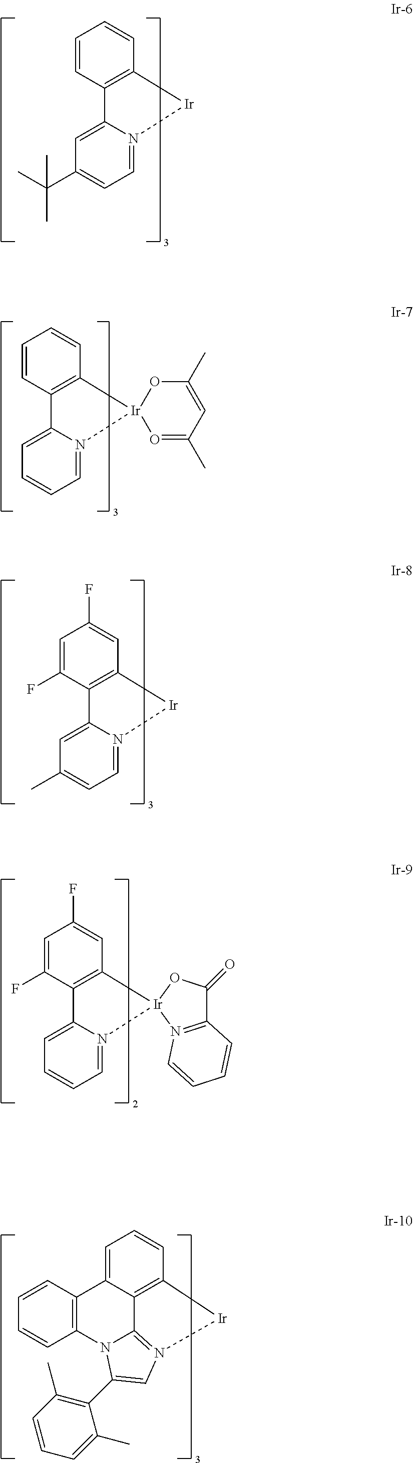

In addition, a fluorescent dopant may be used. Specific examples thereof include fused compounds (e.g., fluorene derivatives, naphthalene derivatives, pyrene derivatives, perylene derivatives, tetracene derivatives, anthracene derivatives, and rubrene), quinacridone derivatives, coumarin derivatives, stilbene derivatives, organic aluminum complexes such as tris(8-quinolinolate)aluminum, organic beryllium complexes, and polymer derivatives such as poly(phenylenevinylene) derivatives, poly(fluorene) derivatives, and poly(phenylene) derivatives.

The electron-injecting material or the electron-transporting material are selected by considering, for example, the balance with the hole mobility of the hole-injecting material or the hole-transporting material. Examples of the material having electron-injecting ability or electron-transporting ability include, but not limited to, oxadiazole derivatives, oxazole derivatives, pyrazine derivatives, triazole derivatives, triazine derivatives, quinoline derivatives, quinoxaline derivatives, phenanthroline derivatives, and organic aluminum complexes.

As the constituent material of the anode, a material having a higher work function is used. Examples thereof include simple metals such as gold, platinum, silver, copper, nickel, palladium, cobalt, selenium, vanadium, and tungsten and alloys thereof; and metal oxides such as tin oxide, zinc oxide, indium oxide, indium tin oxide (ITO), and indium zinc oxide. In addition, electrically conductive polymers such as polyaniline, polypyrrole, and polythiophene also can be used. These electrode materials may be used alone or in combination. The anode may be a monolayer or a multilayer.

On the contrary, as the constituent material of the cathode, a material having a lower work function is used, and examples thereof include alkali metals such as lithium and cesium; alkaline earth metals such as calcium; simple metals such as aluminum, titanium, manganese, silver, lead, and chromium; and alloys of combinations of these simple metals, such as magnesium-silver, aluminum-lithium, and aluminum-magnesium. In addition, metal oxides such as indium tin oxide (ITO) can be used. These electrode materials may be used alone or in combination. The cathode may be a monolayer or a multilayer.

In the organic light-emitting device according to the embodiment, a layer containing the organic compound according to the present invention and layers of other organic compounds are formed by the following methods. In general, the layers are formed by vacuum deposition, ionized vapor deposition, sputtering, plasma coating, or known coating (e.g., spin coating, dipping, a casting method, an LB method, or an ink-jetting method) of compounds dissolved in appropriate solvents. In the cases of vacuum deposition, solution coating, or the like, crystallization hardly occurs, and the resulting layer is excellent in storage stability. In addition, in the coating, a film can be formed in a combination with an appropriate binder resin.

Examples of the binder resin include, but not limited to, polyvinyl carbazole resins, polycarbonate resins, polyester resins, ABS resins, acrylic resins, polyimide resins, phenol resins, epoxy resins, silicone resins, and urea resins. These binder resins may be used alone as a homopolymer or a copolymer or in a combination of two or more thereof. In addition, known additives such as a plasticizer, an antioxidant, and a UV absorber may be optionally contained in the layers.

The organic light-emitting device according to the embodiment can be used as a structural member of a display apparatus or a lighting system. Other application includes exposure light sources of electrophotographic image forming apparatuses and backlights of liquid crystal display apparatuses.

The display apparatus includes the organic light-emitting device according to the embodiment in a display section. This display section includes a plurality of pixels. This pixel includes the organic light-emitting device according to the present invention and a TFT device as an example of the switching device that is electrically connected to the organic light-emitting device for controlling luminance. In the display apparatus, the anode or the cathode of the organic light-emitting device is electrically connected to the drain electrode or the source electrode of the TFT device. The display apparatus can be used as an image-displaying apparatus of, for example, a PC.

The display apparatus may be an image output apparatus that includes an image input section for inputting information from, for example, an area CCD, a linear CCD, or memory card and outputs the input image to a display section. The display apparatus may have, as a display section of an image pickup apparatus or an ink-jet printer, both an image output function for displaying an image based on image information input from the outside and an input function for inputting information processed into an image as an operation panel. The display apparatus may be used as a display section of a multi-functional printer.

A display apparatus using the organic light-emitting device according to the embodiment will now be described with reference to the drawing.

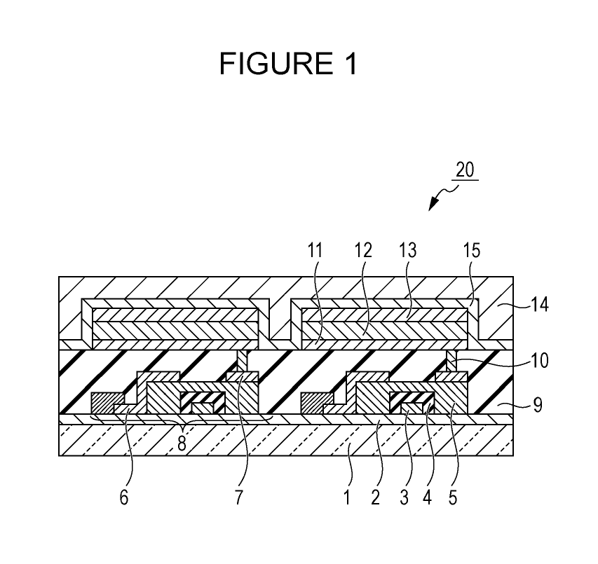

FIG. 1 is a schematic cross-sectional view illustrating an example of a display apparatus having organic light-emitting devices according to the embodiment and TFT devices, an example of switching devices electrically connected to the organic light-emitting devices. FIG. 1 shows two pairs of the organic light-emitting device and the TFT device of a display apparatus 20. The details of the structure of the display apparatus 20 shown in FIG. 1 will be described below.

The display apparatus 20 shown in FIG. 1 includes a substrate 1 such as a glass substrate and a moisture-proof film 2 disposed on the substrate 1 for protecting the TFT devices or the organic compound layer. Reference numeral 3 denotes a metal gate electrode, reference numeral 4 denotes a gate insulating film, and reference numeral 5 denotes a semiconductor layer.

The TFT device 8 includes a semiconductor layer 5, a drain electrode 6, and a source electrode 7. An insulating film 9 is disposed on the TFT device 8. The anode 11 of the organic light-emitting device and the source electrode 7 are connected via a contact hole 10. The display apparatus is not limited to this configuration as long as either the anode or the cathode is connected to either the source electrode or the drain electrode of the TFT device.

In FIG. 1 showing the display apparatus 20, the organic compound layer 12 having a monolayer or multilayer structure is shown as one layer. Furthermore, a first protective layer 14 and a second protective layer 15 are disposed on the cathode 13 in order to prevent deterioration of the organic light-emitting device.

The switching device of the display apparatus according to the embodiment is not particularly limited and may be, for example, a monocrystal silicon substrate, an MIM device, or an a-Si type element.

### EXAMPLES

The present invention will be described in detail with reference to examples, but is not limited thereto.

### Example 1

### Synthesis of Example Compound B8

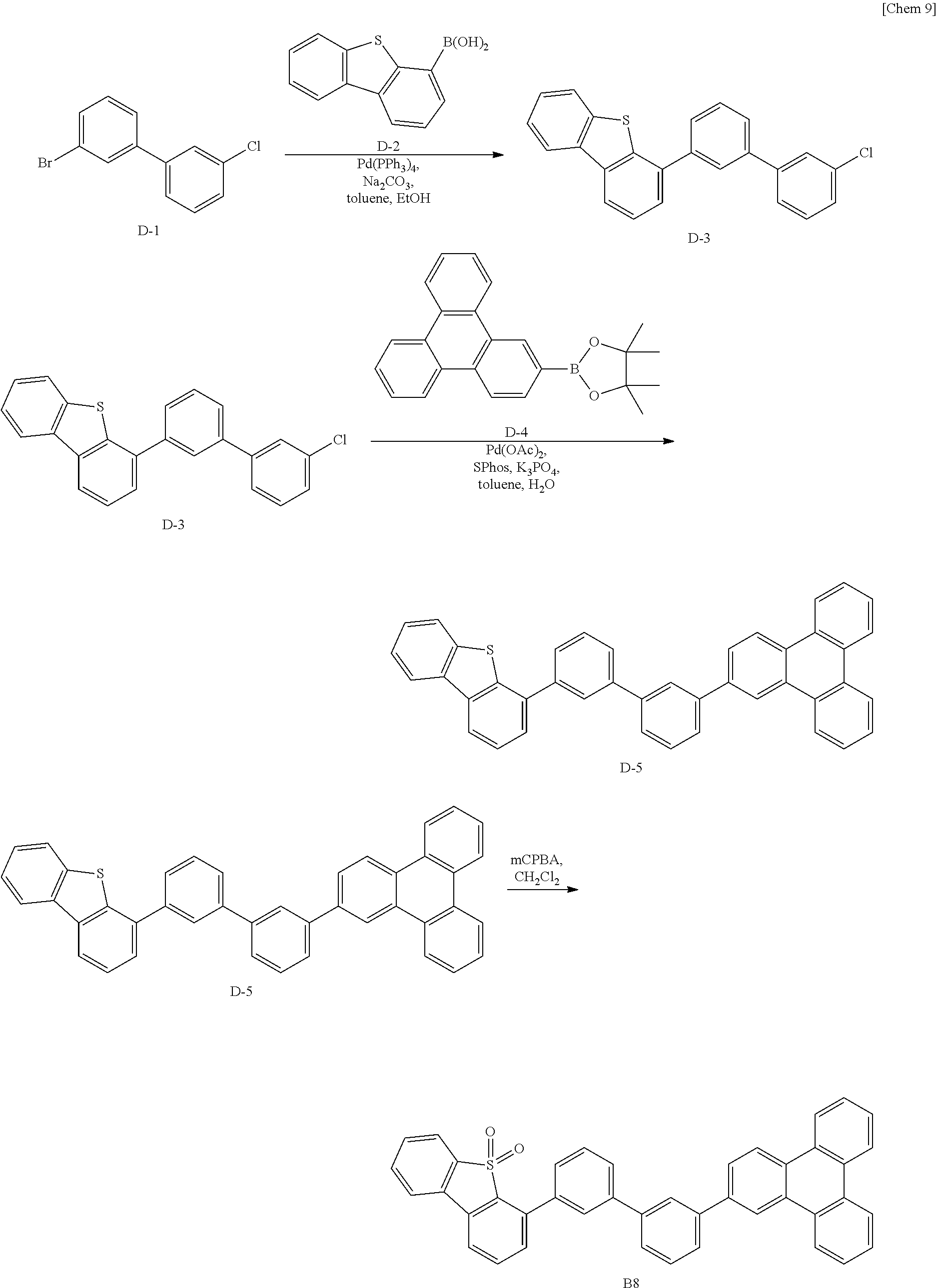

### (1) Synthesis of Compound D-3

The following reagents and solvents:

Compound D-1: 3.00 g (11.2 mmol),
Compound D-2: 2.63 g (11.5 mmol),
toluene: 60 mL,
ethanol: 30 mL, and
an aqueous solution of 10 wt % sodium carbonate: 30 mL were put into a reaction vessel.

Then, tetrakistriphenylphosphine palladium(0) (347 mg, 0.3 mmol) was added to the reaction solution, and the resulting reaction solution was heated to 90° C. and was stirred at the same temperature (90° C.) for 5 hours. Subsequently, the reaction solution was cooled, and water was added thereto, followed by separating extraction. The organic layer was collected and was concentrated under reduced pressure. The resulting residue was purified by silica gel column chromatography (mobile phase: heptane:toluene=20:1) to obtain 2.0 g (yield: 90%) of Compound D-3.

### (2) Synthesis of Compound D-5

The following reagents and solvents:

Compound D-3 (1.62 g, 4.37 mmol),
Compound D-4 (1.78 g, 5.02 mmol),
toluene: 50 mL, and
water: 1.5 mL
were put into a reaction vessel.

Subsequently, the following reagents:

palladium(II) acetate: 80 mg (0.4 mmol),
2-dicyclohexylphosphino-2′,6′-dimethoxybiphenyl (SPhos): 420 mg (1.02 mmol), and
potassium phosphate: 2.75 g (13.0 mmol)
were added to the reaction vessel.

Subsequently, the reaction solution was heated to 120° C. and was stirred at the same temperature (120° C.) for 7.5 hours. After cooling of the reaction solution, water and heptane were added to the reaction solution. The resulting precipitate was collected by filtration and was dissolved in toluene. The resulting toluene solution was applied to silica gel, and the eluted toluene solution was concentrated under reduced pressure. The resulting residue was recrystallized from a solvent mixture of toluene and heptane to obtain 1.91 g (yield: 78%) of Compound D-5.

### (3) Synthesis of Example Compound B8

The following reagent and solvent:

Compound D-5: 800 mg (1.42 mmol), and
dichloromethane: 10 mL
were put into a reaction vessel.

Subsequently, a solution prepared by mixing 876 mg (3.55 mmol) of m-chlorobenzoic acid (mCPBA, purity: 70%) and 10 mL of dichloromethane was added to the reaction vessel. The resulting reaction solution was stirred at room temperature for 3 hours, and then water and chloroform were added thereto, followed by neutralization with an aqueous solution of saturated sodium bicarbonate. Then, extraction with chloroform was performed, and the organic layer was dried over sodium sulfate, followed by drying under reduced pressure. The resulting residue was recrystallized from xylene to obtain Example Compound B8 (470 mg, yield: 56%) as a white solid.

Example Compound B8 was confirmed by mass spectrometry as a peak of M⁺ at m/z 594.

The structure of Example Compound B8 was confirmed by ¹H-NMR.

¹H-NMR (CDCl₃, 400 MHz) σ (ppm): 7.52-7.88 (m, 17H), 8.00 (d, J=8.0 Hz, 1H), 8.26 (s, 2H), 8.67-8.71 (m, 3H), 8.75 (d, J=8.0 Hz, 1H), 8.85 (d, J=8.0 Hz, 1H), 9.01 (s, 1H).

The T₁ energy of Example Compound B8 in a dilute solution of toluene was measured with a spectrophotometer U-3010, manufactured by Hitachi, Ltd. In the measurement, the toluene solution (1×10⁻⁴ mol/L) was cooled to 77K. The phosphorescent component was measured at an excitation wavelength of 350 nm, and the wavelength at the initial rise of the phosphorescence emission spectrum was defined as the T₁ level. The result of the measurement showed a T₁ level of 473 nm.

The HOMO level of Example Compound B8 was measured. Specifically, Example Compound B8 in a thin film form prepared by spin coating of a dilute solution of chloroform containing Example Compound B8 was subjected to measurement of the HOMO level using a photoelectron spectrometer AC-3 (Riken Keiki Co., Ltd.). As a result of the measurement, a HOMO level of −6.47 eV was obtained. The absorption spectrum of Example Compound B8 in the thin film form was measured with a UV/VIS spectrometer V-560 (JASCO Corp.), and the band gap determined from the obtained absorption edge was 3.44 eV. The LUMO level was calculated using the HOMO level and the band gap determined above by the following equation:

[LUMO level]=[HOMO level]+[band gap].

The calculated LUMO level was −3.03 eV.

### Example 2

### Synthesis of Example Compound B2

Example Compound B2 was synthesized as in Example 1 except that Compound D-6 shown below was used instead of Compound D-4 in Example 1(2).

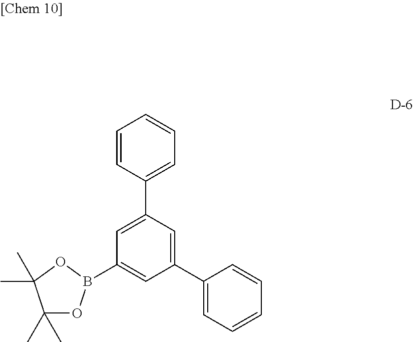 [SMILES: CC1(C)OB(c2cc(-c3ccccc3)cc(-c3ccccc3)c2)OC1(C)C]

Example Compound B2 was confirmed by mass spectrometry as a peak of M⁺ at m/z 596.

### Example 3

### Synthesis of Example Compound B3

Example Compound B3 was synthesized as in Example 1 except that Compound D-7 shown below was used instead of Compound D-4 in Example 1(2).

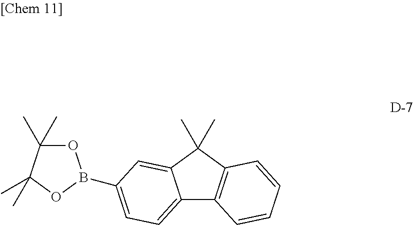 [SMILES: CC1(C)c2ccccc2-c2ccc(B3OC(C)(C)C(C)(C)O3)cc21]

Example Compound B3 was confirmed by mass spectrometry as a peak of M⁺ at m/z 560.

### Example 4

### Synthesis of Example Compound B6

Example Compound B6 was synthesized as in Example 1 except that Compound D-8 shown below was used instead of Compound D-4 in Example 1(2).

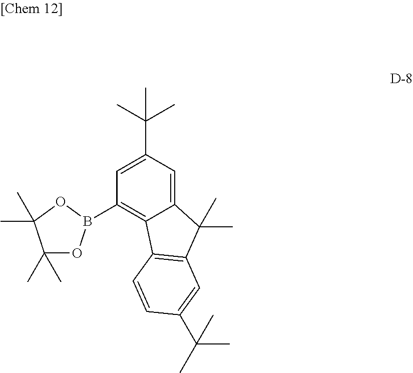

Example Compound B6 was confirmed by mass spectrometry as a peak of M⁺ at m/z 672.

### Example 5

### Synthesis of Example Compound A3

Example Compound A3 was synthesized as in Example 1 except that Compound D-9 shown below was used instead of Compound D-1 in Example 1(1) and that Compound D-6 was used instead of Compound D-4 in Example 1(2).

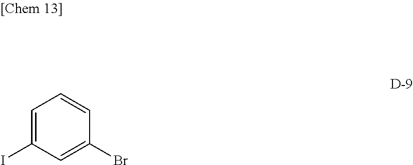

Example Compound A3 was confirmed by mass spectrometry as a peak of M⁺ at m/z 520.

### Example 6

### Synthesis of Example Compound A9

Example Compound A9 was synthesized as in Example 1 except that Compound D-9 was used instead of Compound D-1 in Example 1(1).

Example Compound A9 was confirmed by mass spectrometry as a peak of M⁺ at m/z 518.

### Example 7

### Synthesis of Example Compound A10

Example Compound A10 was synthesized as in Example 1 except that Compound D-9 was used instead of Compound D-1 in Example 1(1) and that Compound D-10 shown below was used instead of Compound D-4 in Example 1(2).

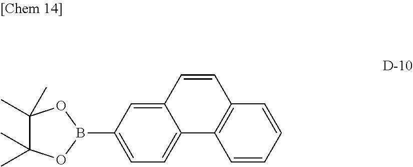 [SMILES: CC1(C)OB(c2ccc3c(ccc4ccccc43)c2)OC1(C)C]

Example Compound A10 was confirmed by mass spectrometry as a peak of M⁺ at m/z 468.

### Example 8

In this Example, an organic light-emitting device in which an anode, a hole-transporting layer, a light-emitting layer, an exciton-blocking layer, an electron-transporting layer, and a cathode were disposed on a substrate in this order was produced by a method shown below. A part of the compounds used in this Example are shown below.

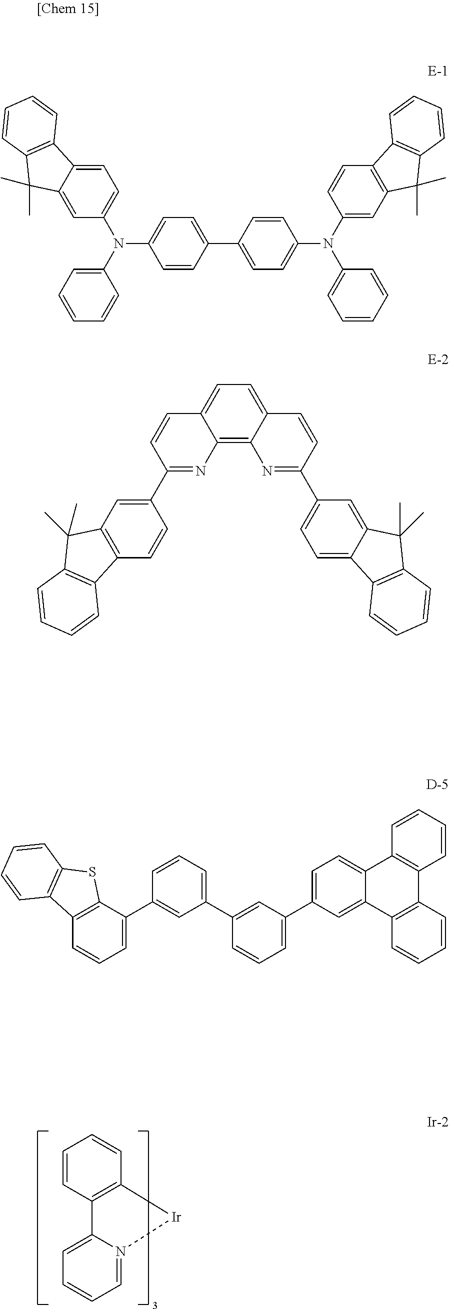

An anode having a thickness of 120 nm was formed by sputtering ITO on a glass substrate. The substrate provided with the ITO electrode (anode) was used as a transparent electrically conductive support substrate (ITO substrate) in the following processes.

On this ITO substrate, organic compound layers and electrode layers shown in Table 3 were successively formed by resistance heating vacuum vapor deposition in a vacuum chamber of 1×10⁻⁵ Pa. On this occasion, the area where the electrodes (metal electrode layer, cathode) facing each other was adjusted to be 3 mm².

TABLE 3

| | Material | Thickness (nm) |
|---|---|---|
| Hole-transporting layer | E-1 | 40 |
| Light-emitting layer | Host material: D-5 | 30 |
| | Guest material: Ir-2 | |
| | (host:guest = 90:10 | |
| | (weight ratio)) | |
| Hole-blocking layer | Example Compound B8 | 10 |
| Electron-transporting layer | E-2 | 0 |
| First metal electrode layer | LiF | 0.5 |
| Second metal electrode layer | Al | 100 |

Then, in order to prevent deterioration of the organic light-emitting device due to absorption of moisture, covering with a protective glass plate and sealing with an acrylic polymer adhesive were performed in a dried air atmosphere to obtain an organic light-emitting device.

A voltage of 5.7 V was applied to the resulting organic light-emitting device using the ITO electrode as the positive electrode and the Al electrode as the negative electrode. As a result, green light emission with a luminous efficiency of 59 cd/A and a luminance of 4000 cd/m² was observed. In this device, the CIE chromaticity coordinate was (x, y)=(0.32, 0.63) to reveal that green light was emitted.

### Example 9

A device was produced as in Example 8 except that the hole-blocking layer was formed using Example Compound B6 instead of Example Compound B8 in Example 8.

A voltage of 6.8 V was applied to the resulting organic light-emitting device using the ITO electrode as the positive electrode and the Al electrode as the negative electrode. As a result, green light emission with a luminous efficiency of 55 cd/A and a luminance of 4000 cd/m² was observed. In this device, the CIE chromaticity coordinate was (x, y)=(0.30, 0.64) to reveal that green light was emitted.

### Example 10

A device was produced as in Example 8 except that the hole-blocking layer was formed using Example Compound A9 instead of Example Compound B8 in Example 8.

A voltage of 6.6 V was applied to the resulting organic light-emitting device using the ITO electrode as the positive electrode and the Al electrode as the negative electrode. As a result, green light emission with a luminous efficiency of 58 cd/A and a luminance of 4000 cd/m² was observed. In this device, the CIE chromaticity coordinate was (x, y)=(0.32, 0.63) to reveal that green light was emitted.

### Comparative Example 1

A device was produced as in Example 8 except that the hole-blocking layer was formed using Compound I-B shown below instead of Example Compound B8 in Example 8.

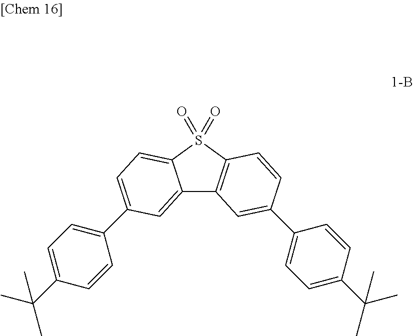 [SMILES: CC(C)(C)c1ccc(-c2ccc3c(c2)-c2cc(-c4ccc(C(C)(C)C)cc4)ccc2S3(=O)=O)cc1]

A voltage of 6.7 V was applied to the resulting organic light-emitting device using the ITO electrode as the positive electrode and the Al electrode as the negative electrode. As a result, green light emission with a luminous efficiency of 40 cd/A and a luminance of 4000 cd/m² was observed. In this device, the CIE chromaticity coordinate was (x, y)=(0.30, 0.62) to reveal that green light was emitted.

As described above, the organic compound (dibenzothiophene dioxide compound) according to the present invention has a high T₁ energy suitable for phosphorescent light-emitting devices that emit blue or green light and also has deep HOMO and LUMO levels. Accordingly, a stable organic light-emitting device having a high luminous efficiency can be obtained by using the organic compound of the present invention as a constituent material of the organic light-emitting device.

While the present invention has been described with reference to exemplary embodiments, it is to be understood that the invention is not limited to the disclosed exemplary embodiment. The scope of the following claims is to be accorded the broadest interpretation so as to encompass all such modifications and equivalent structures and functions.

This application claims the benefit of Japanese Patent Application No. 2011-012531, filed Jan. 25, 2011, which is hereby incorporated by reference herein in its entirety.

### REFERENCE SIGNS LIST

8 TFT device
11 anode
12 organic compound layer
13 cathode

1. A dibenzothiophene dioxide compound represented by the following Formula [1] or [2]: 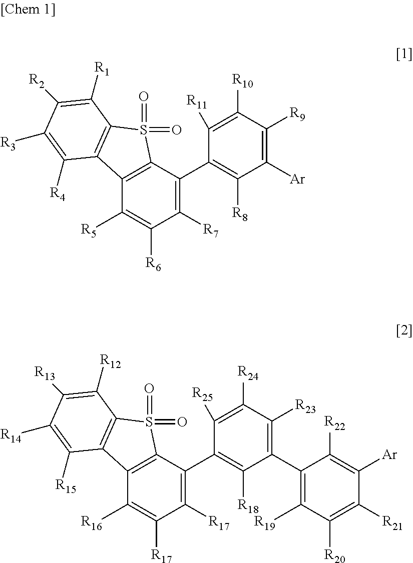
wherein Ar represents a substituent selected from phenyl groups, terphenyl groups, phenanthryl groups, fluorenyl groups, and triphenylenyl groups and optionally includes an alkyl group having 1 to 4 carbon atoms as a substituent;
R₁ to R₁₁ each independently represent a hydrogen atom or an alkyl group having 1 to 4 carbon atoms;
R₁₂ to R₂₅ each independently represent a hydrogen atom or an alkyl group having 1 to 4 carbon atoms.

2. An organic light-emitting device comprising:
an anode and a cathode; and
an organic compound layer disposed between the anode and the cathode and including at least a light-emitting layer, wherein
the organic compound layer contains the dibenzothiophene dioxide compound according to claim 1.

3. The organic light-emitting device according to claim 2, wherein
the organic compound layer further includes a hole-blocking layer containing the dibenzothiophene dioxide compound.

4. The organic light-emitting device according to claim 2, wherein
the light-emitting layer includes a host material and a guest material; and
the host material is composed of two or more materials including the dibenzothiophene dioxide compound.

5. The organic light-emitting device according to claim 2, wherein the light-emitting layer emits phosphorescence.

6. A display apparatus comprising:
a plurality of pixels each having the organic light-emitting device according to claim 2 and a switching device electrically connected to the organic light-emitting device.

7. An image outputting apparatus comprising:
an image input section for inputting an image and a display section for outputting an image, wherein
the display section includes a plurality of pixels each having the organic light-emitting device according to claim 2 and a switching device electrically connected to the organic light-emitting device.

8. A lighting system comprising the organic light-emitting device according to claim 2.

9. An exposure light source of electrophotographic image forming apparatus comprising the organic light-emitting device according to claim 2.

10. An apparatus comprising a substrate and the organic light-emitting device according to claim 2.
